(19) United States
(12) Patent Application Publication
(10) Pub. No.: US 2016/0358700 A1
(43) Pub. Date: Dec. 8, 2016

(54) Electrode Fixing Sleeve Having An Adhesion-Enhancing Surface Structure

(71) Applicant: BIOTRONIK SE & Co. KG, Berlin (DE)

(72) Inventors: Gernot Kolberg, Berlin (DE); Michael Friedrich, Kleinmachnow (DE)

(21) Appl. No.: 15/168,829

(22) Filed: May 31, 2016

(30) Foreign Application Priority Data
Jun. 2, 2015 (DE) 10 2015 108 670.0
Jun. 2, 2015 (DE) 10 2015 108 671.9
Jun. 2, 2015 (DE) 10 2015 108 672.7

(51) Int. Cl. H01B 17/58; A61N 1/05

(52) U.S. Cl. CPC H01B 17/58; A61N 1/05

(57) ABSTRACT

An electrode fixing sleeve is provided, an object of which is to create an electrode fixing sleeve, which, when used in a system with an electrode lead, does not damage the electrode lead even when a very strong substantially radial force is exerted onto the electrode fixing sleeve. In accordance with at least this object, an inner lateral surface of the electrode fixing sleeve is provided at least in part with an adhesion-enhancing surface structure. Since the adhesion-enhancing surface structure has adhesion-enhancing properties, this provides the advantage that the electrode fixing sleeve can be secured without exerting a strong radial force onto an electrode lead.

### CROSS-REFERENCE TO RELATED APPLICATIONS

This patent application claims the priority of co-pending German Patent Application Nos. DE 10 2015 108 672.7; DE 10 2015 108 670.0; and DE 10 2015 108 671.9, all filed on Jun. 2, 2015 in the German Patent Office, the disclosures of which are hereby incorporated by reference in their entireties.

### TECHNICAL FIELD

The present invention relates to an electrode fixing sleeve having an adhesion-enhancing surface structure. The present invention also relates to a system consisting of the electrode fixing sleeve and an electrode lead.

### BACKGROUND

Cardiovascular disorders are some of the most serious diseases of modern society. Many disorders nowadays are still fatal. Older people, in particular, suffer from cardiovascular disorders. In view of the rising life expectancy and the growing number of chronic cardiac disorders, a further increase in these diseases is therefore to be anticipated. Repeatedly cited primary causes include widespread influencing factors of a modern and globally networked society, such as, stress, smoking, and consumption of too much fat along with the associated excess of weight or hypertension. Genetic disposition or viral infections, however, may also cause cardiac disorders. Since disturbances of the cardiovascular system manifest themselves already at a younger age, and younger people in particular grow up from the start in an environment promoting these influencing factors, a further strengthening of the trend towards cardiovascular disorders is to be anticipated. A key approach is thus constituted by a consequent improvement in medical care.

This leads on the one hand to rising costs and on the other hand to higher demands on the reliability of medical products, for example, cardiac pacemakers, since an increasing number of these systems will likely be in circulation in the future and the reliability in respect of faults must be high, accordingly.

Due to an inhomogeneous contraction of the individual areas of the left ventricle, a cardiac failure, also referred to as cardiac insufficiency, may result. The main cause of inhomogeneous or asynchronous contraction is a fault in the conduction system of the heart. The contraction of the left ventricle can be made homogeneous again or, in simplified terms, can be resynchronized, by means of what is known as cardiac resynchronization therapy, or CRT stimulation for short, and the pumping force of the heart can be regained. The method requires the use of two electrodes. One electrode is implanted in the right ventricle. The target vessel for the electrode relevant to the left ventricle is the coronary sinus. This designates the vessel that conducts venous blood from the coronary arteries into the right atrium. The electrode is implanted in a side branch of the coronary sinus and rests on the outer side of the left ventricle. Due to the simultaneous impulse delivery via both electrodes, the left part of the heart is electrically excited again synchronously and a homogeneous contraction is possible.

The electrical leads are secured to the electrodes via what are known as electrode fixing sleeves (EFSs). These are understood to be special fixing sleeves that are arranged fixedly at a defined point on the electrode lead and are then secured at suitable points in the human body, for example, by being sewn on or in the region of a muscle or a vessel. In the case of newer variants, special fixing sleeves are used that are compressed or screwed in place and then form a fixed unit with the electrode. Electrode fixing sleeves of this type are often produced from a soft plastics material, are slid over the electrode lead, and are then pressed on by means of a sewing thread, also referred to as a ligature. The electrode is fixed axially on the electrode lead due to the resultant static friction. The electrode fixing sleeve can then be sutured at a suitable point in the body.

German Patent No. DE 82 24 292 U1 describes an implantable lead having at least one electrical conductor, which is insulated by a biocompatible sheathing and at the distal end of which an electrode is secured. A lead-fastening means is provided at the proximal end of the conductor and is configured as an anchoring sleeve that surrounds the sheathing and is mounted displaceably on the sheathing. The anchoring sleeve has a peripheral groove, with which the anchoring sleeve can be sutured by means of a thread.

So that a conventional electrode fixing sleeve is fixed on an electrode lead with the necessary axial holding force, the ligature thread must be pulled very tight. The electrode lead may be affected by the radial force acting here on said electrode lead. The insulation material or the sheathing is thus squeezed, and the internal electrode structure compressed. In the worst-case scenario, this may lead to a breakage of the electrode in the region of the electrode fixing sleeve.

The present invention is directed toward overcoming one or more of the above-mentioned problems.

### SUMMARY

An object of the present invention lies in creating an electrode fixing sleeve of the type mentioned in the introduction, which, when used in a system with an electrode lead, has no negative effect on the electrode lead even when the electrode fixing sleeve is subjected to a very strong substantially radial force.

At least the above-stated object is achieved by an item having the features of Claim 1. A technical teaching for organizing a system according to Claim 9 is also disclosed, the elements of said system being the electrode fixing sleeve and an electrode lead.

What is provided is an electrode fixing sleeve, wherein in accordance with the present invention an inner lateral surface of the electrode fixing sleeve is provided at least in part with an adhesion-enhancing surface structure. Since the adhesion-enhancing surface structure itself has adhesion-enhancing properties, this provides the advantage that the electrode fixing sleeve can be secured without exerting a strong radial force onto an electrode lead. A known cause of damage to the electrode lead is thus advantageously eliminated.

The electrode fixing sleeve is configured in such a way that the adhesion-enhancing surface structure is disposed closely enough to the surface structure of the electrode lead when the electrode fixing sleeve is arranged on the electrode lead. A diameter of the inner lateral surface of the electrode fixing sleeve, to which the adhesion-enhancing structure is applied, is preferably such that when the electrode fixing sleeve is arranged concentrically with the electrode lead a radial gap between the inner lateral surface of the electrode fixing sleeve and an outer surface of the electrode lead is provided which lies within a permissible range. In the context of the present invention, the inner lateral surface corresponds at least in portions to an inner surface of a hollow cylinder. The electrode lead of the described system preferably also has a rotationally symmetrical design, in particular, having a circular cross section. As a result, the diameter of the inner lateral surface of the electrode fixing sleeve is system-dependent and is dependent on an outer diameter of the electrode lead. It is therefore expedient to specify a parametric design specification for the diameter of the inner lateral surface of the electrode fixing sleeve, since a person skilled in the art otherwise would not be able to carry out the subject matter of the present invention. The design specification is as follows:

D=d+2·R (Eq. 1)

wherein D designates the diameter of the inner lateral surface of the electrode fixing sleeve, d designates a freely selectable parameter corresponding to the outer diameter of the electrode lead, and R corresponds to the radial gap. The permissible value range for the radial gap R is between 10 μm and 1500 μm, more preferably between 20 μm and 750 μm, and particularly preferably between 50 μm and 300 μm. The radial gap R and the freely selectable parameter d are system-dependent variables. In other words: R is provided first by a formation of the system, consisting of the electrode fixing sleeve and the electrode lead, when d and D are defined beforehand, or in other words, an advantageous value of the diameter D is provided from the design specification when d is freely selected and a preferred value is used for R.

The adhesion-enhancing structure is preferably formed as a gecko structure. The present invention thus utilizes an alternative possibility for the connection of different surfaces via the phenomenon of dry adhesivity. Dry adhesivity is understood in the present case to be the formation of adhesive forces between surfaces without adhesion-enhancing substances, such as, for example, glues. Adhesion systems of this type are also known, for example, from nature, for example in the case of gecko legs or insect legs. It is assumed that in such systems the adhesion forces are based on van-der-Waals forces. The adhesion-generating surface for this purpose has an adhesion-enhancing surface structure, for example, a multiplicity of brush-like or hair-like elements, which lead to a very large increase in the available contact area. With the enlargement of the contact area, the strength of the adhesion forces formed in the event of contact consequently also increases. The use of adhesion-enhancing surface structures of this type is proposed for example by Alborz Mandavi et al., ‘A Biodegradable and Biocompatible Gecko-inspired Tissue Adhesive’, PNAS (2008), Vol. 105, No. 7, 2307-2312. In the case of dry adhesivity, the strength of the adhesion between two surfaces is therefore related to the area available for the adhesion. Two similar surfaces, for example, an inner and an outer lateral cylindrical area or two planar areas, adhere much better to one another than, for example, a planar surface and a spherical surface. Generally, the greater is the area available for adhesion, the better two surfaces will adhere to one another.

As a result, the electrode fixing sleeve according to the present invention may also be an element in alternative systems that, as a further element, have an alternative joining partner, for example, a cable, a tube, or the like. In principle, all joining partners are suitable for having a surface form matching the inner lateral surface of the electrode fixing sleeve. Where, in the context of the present invention, statements are made hereinafter on the basis of the example of the electrode lead, this in no way implies that said statements are limited to the electrode lead.

The adhesion-enhancing surface structure may have between 10 and 1,000,000 rods per square millimeter, for example. The ratio of diameter and length of the rods may be between 1:2 and 1:2,000. The cross section of the rod may be cross-profiled, for example, completely or partially round, triangular, rectangular, square or internally hollow. It may have a T-profile or may correspond to a crescent-shaped outline. A preferred bending direction of the rod can thus be predefined. Alternatively, or in combination, the rods can be pre-bent or obliquely attached. A uniform bending direction of the rods may prevent the rods from becoming entangled with one another. The rods may also have a longitudinal profile. They may thus be thickened at the root, where they bear against the component to be fixed, and may taper toward the end.

The adhesion-enhancing surface structure can also consist of rods that branch out. The end of the last branch can be thickened again. The greatest extent of the thickened portion corresponds at most to 100 times the rod diameter on which the thickened portion sits. The end of the last branch may also be planar or rounded or pointed. A lobe-like structure, similarly to a scoop, can be located at the end of the last branch and is attached at one end. The lobe-like structure is preferably attached at one end to the rods in such a way that the angle of the rods is continued. In the event of a transverse force of the component in the detaching direction (for example, in an anticlockwise direction), the lobe-like structure peels away from the tissue, which significantly facilitates the detachment, whereas in the event of transverse force in the other direction only a shear force is caused, which not only does not detach the fixing, but aids the fixing.

The fixing and detachment forces can be set by organization of the bending direction of the rods on the surface. The structures are fixed particularly well when as many rods as possible absorb the tensile forces simultaneously. If the fixing is to be released, the rods must be individually loaded, where possible, so as to enable a detachment even with low forces. Due to the preferred bending direction of the rods, a force acting laterally on the component can be converted into a tensile force or into a compressive force, depending on direction. A force against the rod orientation leads to a force compressing the rod, which causes the rod to bend, as a result of which a rolling motion occurs at the fixing surface, which peels off the fixing surface. This effect can also be utilized over a number of rod sections. For example, only the lower end of the rods may thus be provided with a preferred direction. The subsequent, for example, branched structures are peeled off. An equivalent effect is attained when the rods do not have a preferred bending direction, but are already obliquely attached or pre-curved.

A special embodiment of the rods, which are pre-bent or provided with a preferred bending direction, is one in which the rods are pre-bent about a pivot point, preferably the point of the electrically active or sensitive area in one direction, preferably in an anticlockwise direction. A rotation at the component in an anticlockwise direction rolls each individual rod end about the fixing point and peels it off. The fixing can thus be provided by pressing the component on or by rotation in a clockwise direction. Detachment occurs by rotation in an anticlockwise direction.

Besides the specified tangential orientation of the rods, further structured arrangements are conceivable, for example, an area in which the rods point in one direction is detachable by a force in this direction and is stable in the other direction.

If the component is to be detached by means of an orthogonally acting force, it is expedient for the fixing area to be designed as a membrane and for the rods to point towards the center point of the membrane. If the component is removed perpendicularly, the rods detach from the outside in. This process can be triggered alternatively by a ram, which presses from the inside onto the membrane, or by fluid pressure.

The adhesion-enhancing surface structure can be manufactured in principle from any material that can be connected to the further constituents of the electrode fixing sleeve and that is sufficiently compatible for an intracorporeal use. The adhesion-enhancing surface structures preferably consist of a polymer material, in particular, a silicone. Further possible materials for the structures include, for example, carbon materials, such as, carbon fibers or nanotubes, polypropylene, polytetrafluoroethylene (PTFE), ethylene tetrafluoroethylene (ETFE), polycarbonate, polystyrene, polylactides, for example PDLLA, synthetic spider silk, polyurethanes and copolymers thereof, polyimide, polyamide, polyether ether ketone (PEEK), polysulfone, polyethylene, polyoxymethylene (POM), polyether block amide, chitin, collagen, cellulose, keratin, metals, glass, and ceramic.

An adhesion-enhancing surface structure can be produced by different methods. By way of example, negative molds can be produced by lithographic methods, such as electron beam lithography and laser lithography, or by etching methods. In a subsequent casting method, the positive surface with hair-like extensions is then produced starting from the negative mold (for example, see A. K. Geim et al., Nature Mater. 2, 461-463 (2003) and H. Lee, B. P. Lee and P. B. Messersmith, Nature 448, 338-341 (2007)).

In a preferred embodiment of the present invention, the electrode fixing sleeve comprises at least one inner hollow body and at least one outer hollow body, which are operatively connected to one another, wherein the inner hollow body is provided with the adhesion-enhancing surface structure. This, in particular, provides the advantage that the inner hollow body can be produced with the adhesion-enhancing surface structure in a simple manner, for example, from a cast part. The inner and the outer hollow body preferably have the form of a hollow cylinder. Further embodiments having in inner lateral surface that is cylindrical at least in portions are likewise preferred, wherein these can be derived by a person skilled in the art from the known geometric forms. An operative connection between the inner and the outer hollow body means, in this context, that a variable acting on the inner or outer hollow body, in particular a mechanical force, is transferred to the other hollow body. A transfer function can preferably be selected such that the transfer does not occur one-to-one. The inner hollow body can preferably be arranged in a shaped recess of the outer hollow body and can thus be positioned via its outer surfaces in the outer hollow body. The inner lateral surface of the inner hollow body is preferably provided with the adhesion-enhancing surface structure.

In a further preferred embodiment, the inner and/or the outer hollow body have/has elastic properties. This provides the advantage that the diameter of the inner lateral surface(s) of the inner and/or the outer hollow body can be changed by elastic deformation, and the electrode fixing sleeve can thus be detachably secured without the need for a complex mechanical structure. The generation of elastic properties can be achieved in many ways known to a person skilled in the art. The elastic properties are preferably produced as a result of the selection of an elastic material, for example, an elastomer, or by the creation of elastic structures, which, for example, can be realized by thin wall thicknesses or high length-to-width ratios of a component part.

It is clear from this embodiment of the electrode fixing sleeve that this may have a fixed and a non-fixed state in the system. Here, the fixed state describes the state in which the adhesion-enhancing surface structure enables a flow of force through the electrode lead and the electrode fixing sleeve, in other words the electrode fixing sleeve is secured on the electrode lead. The non-fixed state describes the exact opposite of the fixed state. In the non-fixed state, a flow of force through the electrode lead and the electrode fixing sleeve therefore is not possible, or the electrode fixing sleeve is not secured on the electrode lead. By means of elastic deformation, or formation of the electrode fixing sleeve, it is possible to change between the states. For this purpose, the electrode fixing sleeve is subjected, for example, to a suitable force, the loading line of which extends substantially radially to the electrode fixing sleeve, or orthogonally to the longitudinal axis of the electrode fixing sleeve. When reference is made to force or forces, one or more force pairs is/are therefore generally meant, of which the loading lines are collinear and of which the directions of action are opposite, wherein the forces have the same magnitude.

In a further preferred embodiment, the inner hollow body of the electrode fixing sleeve has at least one slot, which is configured to enable and/or to limit an elastic deformation of the inner hollow body. This provides on the one hand the advantage that the used material can be freely selected, and on the other hand the advantage that the elastic behavior of the inner hollow body can be qualitatively and quantitatively designed, by forming the slot accordingly. The slot must, in particular, be fashioned such that a minimum diameter at the inner lateral surface of the inner hollow body corresponds to the diameter D by limitation of the elastic deformation. A limitation of the elastic deformation of the inner hollow body is preferably provided by effective area pairing. The effective areas may preferably be arranged in a region of the slot. The effective areas can particularly preferably be one or more geometric interfaces of the slot.

In a further preferred embodiment, the electrode fixing sleeve has a means for limiting the effect of a force exerted onto the electrode fixing sleeve on the inner hollow body. This provides the advantage that the inner hollow body having the adhesion-enhancing surface structure cannot be damaged by a strong effective force. The means can preferably be provided as a further hollow body, in particular, as a slotted hollow cylinder, which is arranged between the inner and the outer hollow body and is operatively connected thereto. The slotted hollow cylinder preferably consists of a loadable, preferably metal material, for example, stainless steel. The slot in the hollow cylinder enables an elastic deformation of the further hollow body or of the slotted hollow cylinder. Due to the operative connection to the inner and outer hollow body, all three hollow bodies are thus elastically deformed by an exerted force. This leads to a diameter reduction of all three hollow bodies. The slot in the further hollow body or the slotted hollow cylinder must be formed, in particular, such that a minimum diameter at the inner lateral surface of the inner hollow body corresponds to the diameter D by limitation of the elastic deformation. The elastic deformation of the slotted hollow cylinder is preferably limited by effective area pairing. The effective areas can be arranged preferably in a region of the slot. The effective areas can particularly preferably be one or more geometric interfaces of the slot. The minimum diameter of the inner lateral surface of the further hollow body, preferably of the slotted hollow cylinder, correlates here to the minimal admissible diameter of an inner lateral surface of the inner hollow body, which corresponds to the diameter D. If the force on the electrode fixing sleeve is increased further, a flow of force passes through the paired effective areas in the region of the slot of the slotted hollow cylinder, such that no further an elastic deformation occurs. The effect of the force exerted onto the electrode fixing sleeve on the inner hollow body is thus advantageously limited, and damage caused by the effect of the force is advantageously prevented.

In a further preferred embodiment, the means may also consist of two individual identically formed bodies, which each have the form of a rotationally symmetrical sub-body. A body of this type is obtained when a rotary shape removal is provided over less than 180° about an axis of rotation, wherein the rotating base area has a rectangular shape. If two bodies of this type are provided, these can be considered as two incomplete halves of a hollow cylinder. The two incomplete halves can preferably be arranged between the inner and the outer hollow body and can be operatively connected thereto. The axis of rotation of the two incomplete halves is preferably arranged collinearly to the longitudinal axis of the inner and the outer hollow body. The two bodies, i.e., the incomplete halves, preferably consist of a loadable, preferably metal material, for example, stainless steel. An elastic deformation of the inner hollow body as a result of a force exerted onto the electrode fixing sleeve is then limited by effective area pairing at the rectangular faces of the two bodies. The two bodies are preferably designed in such a way that an undershoot of the minimum diameter of the inner lateral surface of the inner hollow body, which corresponds to the diameter D, is avoided. In the non-elastically deformed state of the electrode fixing sleeve, the distance between the rectangular faces of the two bodies, which are arranged between the inner and the outer hollow body and the axis of rotation of which is arranged collinearly to the longitudinal axis of the inner and of the outer hollow body, thus correlates to the maximum permissible elastic deformation of the inner hollow body, or to the maximum permissible inner diameter reduction thereof.

The active principle of the means for limiting the effect of a force exerted onto the electrode fixing sleeve on the inner hollow body is preferably based on the principle of a form fit. The same is true for the limitation of the elastic deformation of the inner and outer hollow body. A large number of further possible embodiments are thus conceivable and will be implemented by a person skilled in the art as necessary.

In a further preferred embodiment, the electrode fixing sleeve has at least one peripheral groove for accommodating a ligature element. This provides the advantage that fastening means, such as, for example, ligature threads can be used, which are known in the medical field and are usually available. The force required for the securing, or elastic deformation, of the electrode fixing sleeve can be applied with the aid of such ligature elements. The electrode fixing sleeve preferably has two or three peripheral grooves for accommodating ligature elements. The at least one peripheral groove is also preferably configured to accommodate alternative securing elements, such as, for example, a clamp, a clip, or a ratchet. Where appropriate, the securing is reversible with the aid of the securing elements.

In a further preferred embodiment, the inner lateral surface provided by the inner hollow body has an oval contour, or an oval cross-sectional shape, in cross section relative to the longitudinal axis of the electrode fixing sleeve in the fixed state of the electrode fixing sleeve. In this embodiment, the fixed state is provided when there is no elastic deformation of the electrode fixing sleeve, and this is arranged concentrically with the electrode lead. The adhesion-enhancing surface structure is arranged in at least two regions of the inner lateral surface, extending from two points. The two points are the points of the inner lateral layer that, in the fixed state, lie closest to the longitudinal axis of the electrode fixing sleeve in the cross section of the electrode fixing sleeve. In other words, if, in the fixed state, a virtual circle is placed concentrically in the cross-sectional shape of the electrode fixing sleeve, the two points of the inner lateral layer lying closest to the longitudinal axis of the electrode fixing sleeve contact said circle tangentially when the circle has the diameter D or, in other words, the two points of the inner lateral layer lying closest to the longitudinal axis of the electrode fixing sleeve lie, in the fixed state, on a virtual circle arranged concentrically with the longitudinal axis of the electrode fixing sleeve, wherein a plane in which the circle lies is intersected normally by the longitudinal axis of the electrode fixing sleeve. If the electrode fixing sleeve is to be brought into the non-fixed state, this can be implemented preferably by elastic deformation. For this purpose, a force is exerted onto the electrode fixing sleeve, for example, the loading line of said force being applied collinearly to a longer transverse axis of the oval cross-sectional shape. Both the inner and the outer hollow body preferably have resilient properties. The inner hollow body is elastically deformed by the exerted force and is brought from an oval cross-sectional shape into a substantially circular cross-sectional shape. The two points are thus distanced from the longitudinal axis of the electrode fixing sleeve. This embodiment of the present invention provides the advantage that a force must be applied only when the electrode fixing sleeve is to be removed. The electrode fixing sleeve can thus be removed, slid on the electrode lead, and fixed again in a simple manner.

The outer surface of the electrode fixing sleeve can preferably be provided with two cavities, which are oriented along the longer transverse axis of the oval cross-sectional shape, such that the sleeve is removed when it is squeezed via these cavities between a thumb and index finger.

A method for securing an electrode lead is also disclosed, wherein a frictionally engaged connection is produced between an electrode lead and a securing element. In accordance with the present invention, a defined gap and/or a defined pressing force are/is produced in a contact region of the securing element, between said element and the electrode lead. In accordance with the present invention, surface structures of the securing element are thus guided so closely against a surface of the electrode lead in the contact region that a connection is produced.

Further embodiments, features, aspects, objects, advantages, and possible applications of the present invention could be learned from the following description, in combination with the Figures, and the appended claims.

Further preferred embodiments of the present invention are provided by arbitrary advantageous combination of the features specified in the dependent claims and in the following description.

### DESCRIPTION OF THE DRAWINGS

The present invention will be explained in greater detail hereinafter on the basis of an exemplary embodiment and associated drawings. In the Figures:

FIG. 1 shows a preferred exemplary embodiment of an electrode fixing sleeve according to the present invention in the non-fixed state as an exploded illustration (without electrode lead).

FIG. 2 shows a preferred exemplary embodiment of an electrode fixing sleeve according to the present invention in the fixed state as a sectional view and as a view along a longitudinal axis (without electrode lead).

FIGS. 3A-3B show alternative embodiments of an inner hollow body.

FIG. 4 shows alternative variants of a means for limiting the effect of a force, exerted onto the electrode fixing sleeve, on an inner hollow body.

FIG. 5 shows a cross-sectional view of an electrode fixing sleeve according to the present invention in the fixed state with an oval cross-sectional shape of an inner lateral surface of the inner hollow body in an alternative embodiment.

### DETAILED DESCRIPTION

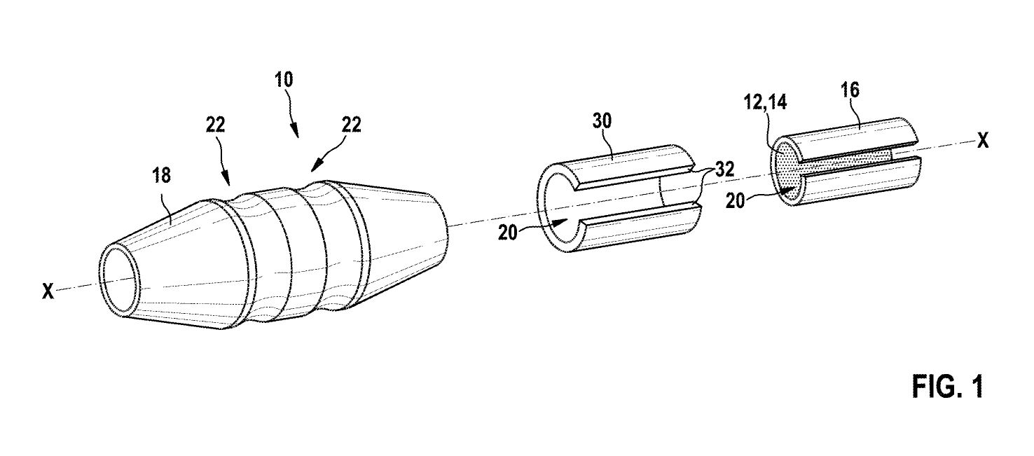

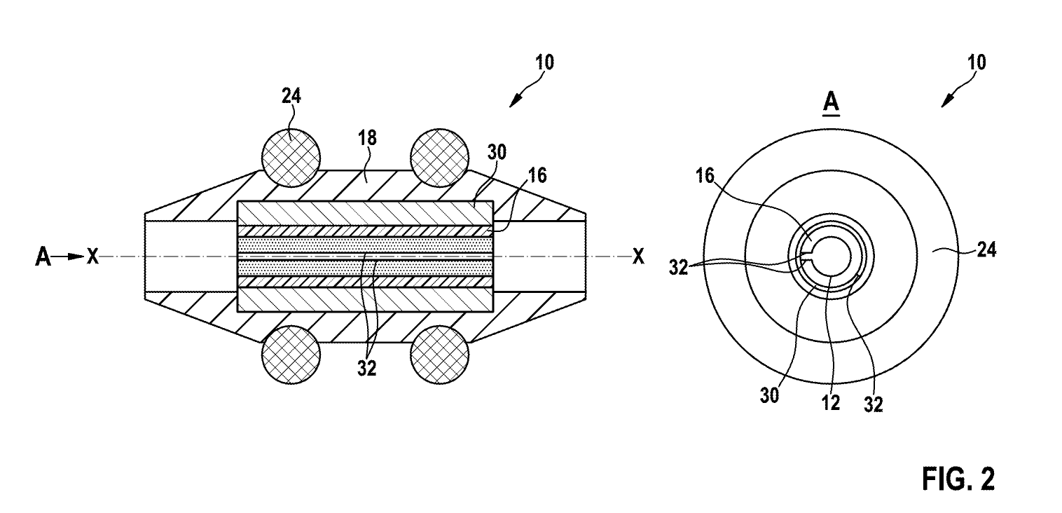

FIGS. 1-2 show a preferred exemplary embodiment of an electrode fixing sleeve 10 according to the present invention in a non-fixed state as exploded illustration and as a sectional view and in a view along a longitudinal axis X of the electrode fixing sleeve 10. The electrode lead is not illustrated.

In accordance with the present invention, the electrode fixing sleeve 10 has an inner lateral surface 12, which is provided with an adhesion-enhancing surface structure 14. The electrode fixing sleeve 10 also has two peripheral grooves 22 for accommodating ligature elements 24. The adhesion-enhancing surface structure 14 is formed as a gecko structure. The electrode fixing sleeve 10 comprises an inner hollow body 16 and an outer hollow body 18 and also means for limiting the effect of a force exerted onto the electrode fixing sleeve 10 on the inner hollow body 16, these being operatively connected to one another. The inner hollow body 16 is provided with the adhesion-enhancing surface structure 14.

The means for limiting the effect of a force exerted onto the electrode fixing sleeve 10 on the inner hollow body 16 is formed as a hollow cylinder 30 having a slot 20 formed continuously along the longitudinal axis X. The slotted hollow cylinder 30 is arranged between the inner hollow body 16 and the outer hollow body 18 and is operatively connected thereto. The slotted hollow cylinder 30 consists of stainless steel. The inner hollow body 16 consists of polyamide and the outer hollow body 18 consists of a silicone. The inner hollow body 16 has a slot 20 formed continuously along the longitudinal axis X. The slots 20 in the hollow cylinder 30 and the inner hollow body 16, as well as elastic material properties of the outer hollow body 18, enable an elastic deformation of the electrode fixing sleeve 10. Due to the operative connection of the slotted hollow cylinder 30 to the inner hollow body 16 and the outer hollow body 18, all three bodies can be elastically deformed by an exerted force. The slot 20 in the hollow cylinder 30 is formed such that, when a minimum diameter D is reached at the inner lateral surface 12 of the inner hollow body 16, the elastic deformation of the slotted hollow cylinder 30 is limited by pairing of the effective areas 32. Since there is also no possibility of any further elastic deformation with a further increase of the force acting on the electrode fixing sleeve 10, the diameter D also does not reduce further. In the system with an electrode lead, a gap R is provided between the inner lateral surface 12 of the inner hollow body 16 and the surface of the electrode lead in the fixed state. The effective areas 32 of the inner hollow body 16 do not contact one another in this case.

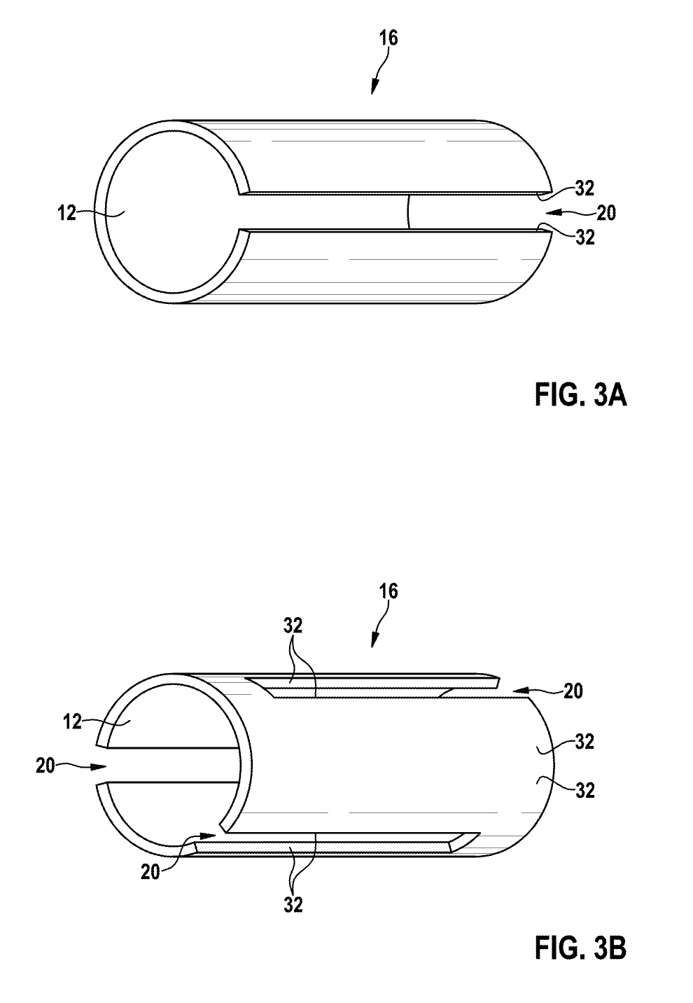

FIGS. 3A-3B show two alternative embodiments of the inner hollow body 16. FIG. 3A shows an inner hollow body 16 having an inner lateral surface 12 and effective areas 32 in a region of the slot 20. The slot is formed continuously over the entire length of the inner hollow body 16. This variant provides the advantage that the elastic deformation occurs uniformly and that the inner hollow body 16 with this slot 20 can be produced easily.

FIG. 3B shows a further embodiment of the inner hollow body 16 with a plurality of slots 20. In this variant, the inner hollow body 16 has three slots 20, which are not formed continuously over the entire length of the inner hollow body 16. Depending on the portion of the length of the inner hollow body 16 over which the slots 20 extend, the resilient properties of the inner hollow body 16 increase or decrease. This provides the advantage that the elastic properties can be accurately set.

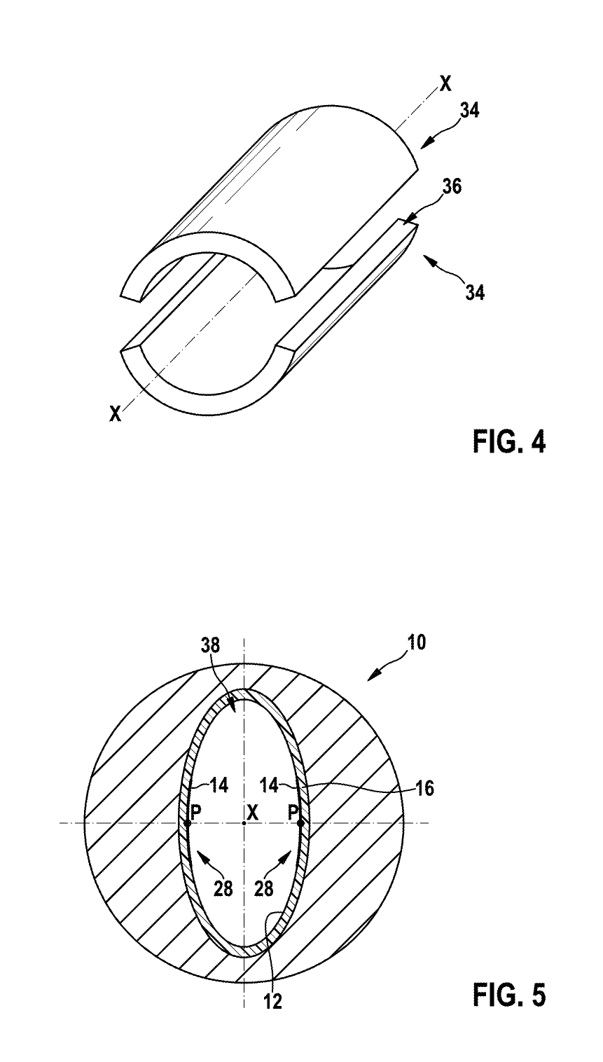

FIG. 4 shows an alternative variant of a means for limiting the force acting on the inner hollow body 16, said force being produced by exertion of a force onto the outer hollow body 18 of the electrode fixing sleeve 10. The means consists of two individual, identically formed bodies 34, which each have the form of a rotationally symmetrical sub-body. The bodies 34 can be described in an abstract manner as the product of a rotary shape removal through less than 180° about an axis of rotation that, at the same time, forms a longitudinal axis X of the bodies 34. The surface of revolution or base area 36 forming the basis of the rotary shape removal has a rectangular shape. The two bodies 34 constitute two incomplete halves of a hollow cylinder. The two bodies 34 consist of stainless steel. Each of the bodies 34 has two effective areas 32, which correspond to the base area 36 of the rotation removal. Geometric modifications of the effective areas 32 can be made in order to provide more advantageous properties in the case of an effective area pairing.

FIG. 5 shows a cross-sectional view of an electrode fixing sleeve 10 according to the present invention in a fixed state with an oval cross-sectional shape 38 of an inner lateral surface 12 of the inner hollow body 16 in an alternative embodiment. The inner lateral surface 12 provided by the inner hollow body 16, in the fixed state of the electrode fixing sleeve 10, has an oval contour, or an oval cross-sectional shape 38, in cross section relative to the longitudinal axis X of the electrode fixing sleeve 10. The fixed state is present in this embodiment when there is no elastic deformation of the electrode fixing sleeve 10. The adhesion-enhancing surface structure 14 is arranged in at least two regions 28 of the inner lateral surface 12, which extend over part of the inner lateral surface 12 from two points P. The two points P are the points of the inner lateral surface 12 lying closest to the longitudinal axis X of the electrode fixing sleeve in the cross section of the electrode fixing sleeve 10 in the fixed state.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An electrode fixing sleeve, wherein an inner lateral surface of the electrode fixing sleeve is provided at least in part with an adhesion-enhancing surface structure.

2. The electrode fixing sleeve according to claim 1, wherein the electrode fixing sleeve comprises at least one inner hollow body and at least one outer hollow body, which are operatively connected to one another, wherein the inner hollow body is provided with the adhesion-enhancing surface structure.

3. The electrode fixing sleeve according to claim 2, wherein the inner and/or the outer hollow body has elastic properties.

4. The electrode fixing sleeve according to claim 3, wherein the inner hollow body has at least one slot, which is configured to enable and/or to limit an elastic deformation of the inner hollow body.

5. The electrode fixing sleeve according to claim 3, wherein the electrode fixing sleeve has a means for limiting the effect of a force exerted onto the electrode fixing sleeve on the inner hollow body.

6. The electrode fixing sleeve according to claim 1, in which the electrode fixing sleeve has at least one peripheral groove for accommodating a ligature element.

7. The electrode fixing sleeve according to claim 3, wherein the inner lateral surface provided by the inner hollow body has an oval contour in cross section relative to the longitudinal axis (X) of the electrode fixing sleeve in a fixed state of the electrode fixing sleeve, wherein the adhesion-enhancing surface structure is arranged in at least two regions of the lateral surface, which extend starting from two points (P) of the inner lateral layer lying closest to the longitudinal axis of the electrode fixing sleeve.

8. The electrode fixing sleeve according to claim 2, wherein the inner hollow body and/or the outer hollow body and/or the adhesion-enhancing surface structure consist/consists of a polymer material.

9. A system, consisting of an electrode fixing sleeve according to claim 1; and an electrode lead, wherein the electrode fixing sleeve is arranged concentrically with the electrode lead and a radial gap R is provided between an inner lateral surface of the electrode fixing sleeve and an outer surface of the electrode lead, said gap being between 10 μm and 1500 μm.

10. The system according to claim 9, wherein said gap is between 20 μm and 750 μm.

11. The system according to claim 9, wherein said gap is between 50 μm and 300 μm.
